(19) United States
(12) Patent Application Publication
(10) Pub. No.: US 2026/0013914 A1
(43) Pub. Date: Jan. 15, 2026

(54) Bone Fixation Plate and System

(71) Applicant: Field Orthopaedics Pty Ltd, Teneriffe, QLD (AU)

(72) Inventors: Kau Shean LIANG, Red Hill QLD (AU); Michael MAURER, Red Hill QLD (AU)

(21) Appl. No.: 18/875,457

(22) PCT Filed: Jun. 16, 2023
(86) PCT No.: PCT/AU2023/050536

§ 371 Date: Dec. 16, 2024

(30) Foreign Application Priority Data
Jun. 17, 2022 (AU) 2022901651

(51) Int. Cl. A61B 17/80; A61B 17/86

(52) U.S. Cl. CPC A61B 17/8057; A61B 17/8605

(57) ABSTRACT

A bone fixation system for reconstruction and/or trauma treatment of bones, the bone fixation plate comprising: a bone fixation plate configured and dimensioned for application to a patient's bone, the bone fixation plate comprising an in-use upper surface and an in-use lower surface with at least one opening formed on the upper and lower surfaces to form a passage extending from the upper surface to the lower surface; each passage being defined by inner walls extending from the upper surface to the lower surface, the inner walls having a height extending between the upper surface and the lower surface, the inner wall further comprising: a threaded inner wall portion extending from the upper surface towards the lower surface, the threaded inner wall portion having threads with a thread pitch (PW); and a non-threaded lower inner wall portion extending from a lower part of the threaded inner wall portion towards the lower surface of the bone fixation plate; and at least one bone screw with a threaded screw head having threads with a thread pitch (PH) for engaging and interfacing with the threaded inner wall for variable angle anchoring of the threaded screw head during use.

### TECHNICAL FIELD

The present invention relates to a bone plate and an associated plate screws for use in repairing bone fractures.

### BACKGROUND

Any references to methods, apparatus or documents of the prior art are not to be taken as constituting any evidence or admission that they formed, or form part of the common general knowledge.

The use of orthopaedic fastening devices, such as bone screws, has greatly aided the medical field in the treatment of bone fractures. More particularly, when treating bone fractures, it is sometimes generally necessary to surgically reposition fragmented bone members in various anatomically acceptable orientations. To fasten the repositioned bone members together bone screws are often used as part of the stabilization process. The use of bone screws can involve the utilisation of an orthopaedic device such as a bone fixation plate that allows attachment of multiple bone screws to a number of bone fragments. During many such surgical interventions, it is beneficial to orient the bone screw at an angle that is non-perpendicular to the bone fixation plate during the stabilization process—for instance, to avoid poor bone stock or fracture lines. Many variable angle locking systems, however, are difficult to manipulate, particularly in terms of variably orienting the screw relative to the fixture. In addition, such locking systems also do not sufficiently prohibit relative motion between the screw and the bone fixation plate to which it is secured.

A number of so-called “polyaxial” bone plate systems are known. Many use a bushing located in a plate hole to lock the degree of screw angulation relative to the plate. In one such system, the bushing is rotatable within the plate hole. A so-called “variable-angle locking” screw is threaded into bone through the bushing and plate hole. As the screw is threaded into bone, the threaded tapered head of the screw engages a threaded internal surface of the bushing to expand the bushing against the inner surface or wall of the plate hole, thereby friction locking the screw at the desired angle relative to the bone plate.

In another known polyaxial bone plate system, a bushing is seated at a desired angle in a plate hole. A fastening screw having an expandable head with a threaded recess is inserted through the bushing and threaded into bone. A locking screw is then threaded into the recess of the screwhead to expand the head outward against the bushing to lock the selected angle of the screw relative to the bone plate.

In still another known polyaxial bone plate system, an expandable ring is positioned in the plate hole. As a bone screw with a tapered head engages the ring and is threaded into bone, the ring expands against the inner surface or wall of the hole to lock the selected angle of the screw relative to the bone plate.

However, these polyaxial bone plate systems have multiple components that can be cumbersome and tedious to manipulate during surgery and more particularly, for example, it is possible that the bushing or expandable ring may pop out during surgery.

In view of the above, it is desirable to provide an improved bone plate systems that comprises lesser number of components and still allows polyaxial or variable angle locking of bone screws.

### SUMMARY OF INVENTION

In an aspect, the invention provides a bone fixation system for reconstruction and/or trauma treatment of bones, the bone fixation system comprising:
a bone fixation plate configured and dimensioned for application to a patient's bone, the bone fixation plate comprising an in-use upper surface and an in-use lower surface with at least one opening formed on the upper and lower surfaces to form a passage extending from the upper surface to the lower surface;
each passage being defined by inner walls extending from the upper surface to the lower surface, the inner walls having a height extending between the upper surface and the lower surface, the inner wall further comprising:
a threaded upper inner wall portion extending from the upper surface towards the lower surface, the threaded inner wall portion having threads with a thread pitch (P_{W});
a non-threaded lower inner wall portion extending from a lower part of the threaded inner wall portion towards the lower surface of the bone fixation plate; and
at least one bone screw with a threaded screw head having threads with a thread pitch (P_{H}) whereby thread profile for the threaded screw head is mismatched relative to thread profile of the threaded upper wall inner portion, wherein the mismatch comprises a gap between threads for the threaded inner wall portion, wherein the gap is greater than a thickness of the threads for the threaded screw head for facilitating engagement of the threaded screw head when interfaced with the threaded upper inner wall portion for effecting variable angle anchoring of the threaded screw head during use, and
   wherein the lower inner wall portion comprises a convergent surface to prevent advancement of the screw head through the passage.

In an embodiment, the gap between the threads for the threaded inner wall region is at least two times the thickness of the threads for the threaded screw head.

In an embodiment, the gap between threads for the threaded inner wall portion is greater than the gap between the threads for the threaded screw head for facilitating engagement of the threaded screw head.

In an embodiment, crest height for threads for the threaded upper inner wall portion is greater than crest height for threads on the threaded head of the bone screw.

In an embodiment, the threaded inner wall portion tapers in a radially inward and downward direction.

Preferably, P_{W}>P_{H} allows the threads on the screw head are sacrificially engaged with the threaded inner wall portion as the screw head is advanced through the passage of the bone plate.

In an embodiment, the passage is defined by a plurality of said inner walls such that adjacently located inner walls are separated by a recessed channel extending radially away from a central axis of the passage.

In an embodiment, the bone plate comprises N number of recessed channels and wherein the N recessed channels are angularly spaced at a distance of 360°/N relative to the central axis of the hole.

In an embodiment, the recessed channels comprise a curved surface.

In an embodiment, the passage further comprises a non-threaded inner wall portion extending from a lower part of the threaded inner wall portion towards the lower surface of the bone fixation plate.

In an embodiment, the non-threaded inner wall portion defines a convergent configuration terminating in an opening with a width that is less than a width of the threaded head of the bone screw for preventing advancement of the screw head resulting in compression between the bone plate and the bone during use.

In an embodiment, the non-threaded wall portion comprises a convergent surface to prevent advancement of the screw head through the passage.

In an embodiment, height of the passage is greater than height of the threaded head of the screw.

In an embodiment, height of the threaded wall portion is greater than height of the threaded head of the bone screw.

In an embodiment, threads for the threaded inner wall have an average thickness that is greater than an average thickness of the threads for the threaded head of the bone screw to facilitate sacrificial engagement of the threaded inner wall portion with the threaded head of the bone screw. Preferably, average thickness for the threaded inner wall is at least twice the average thickness of the threads for the threaded head of the bone screw.

In an embodiment, the passage comprises a vertical axis (V-V), wherein the threaded screw head is configured to lockably mate with the threaded wall portion of the passage at an off axis angle of up to 15 degrees and at least up to 10 degrees in any direction relative to the vertical axis.

In another aspect, the invention comprises a bone fixation plate configured and dimensioned for application to a patients bone for reconstruction and/or trauma treatment of bones, the bone fixation plate comprising: an in-use upper surface and an in-use lower surface with at least one opening formed on the upper and lower surfaces to form a passage extending from the upper surface to the lower surface; each passage being defined by inner walls extending from the upper surface to the lower surface, the inner walls having a height extending between the upper surface and the lower surface, the inner wall further comprising a threaded upper inner wall portion extending from the upper surface towards the lower surface and a non-threaded inner wall portion extending from a lower part of the threaded inner wall portion towards the lower surface of the bone fixation plate; wherein the threaded inner wall portion comprises threads with a thread pitch (P_{W}) adapted for engaging of a threaded screw head having a thread pitch (P_{H}) when interfaced with the threaded upper inner wall portion for effecting variable angle anchoring of the threaded screw head during use, and wherein the lower inner wall portion comprises a convergent surface to prevent advancement of the screw head through the passage.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a top perspective view of a bone fixation plate 100 in accordance with an embodiment.

FIG. 2 is an enlarged view of the bone fixation plate 100.

FIG. 3 is a sectional view of a passage 130 from the bone fixation plate 100.

FIG. 4 is an isolated view of a bone screw 200 to be used with the bone fixation plate 100.

FIG. 5 is a sectional view of the bone screw 200 with the screw 210 shown in engagement with the passage 130 of the bone fixation plate 100.

FIG. 6 is an isolated and enlarged view of the passage opening 130.

FIGS. 7A to 7C show sequential stages of the axial advancement of the bone screw 200 through the passage 130 of the bone fixation plate

### DETAILED DESCRIPTION OF PREFERRED EMBODIMENTS

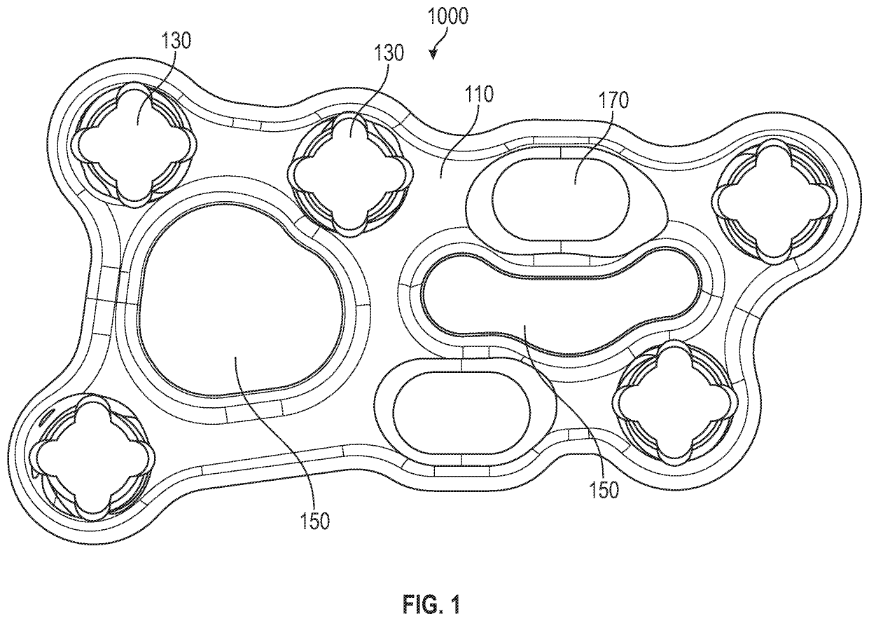

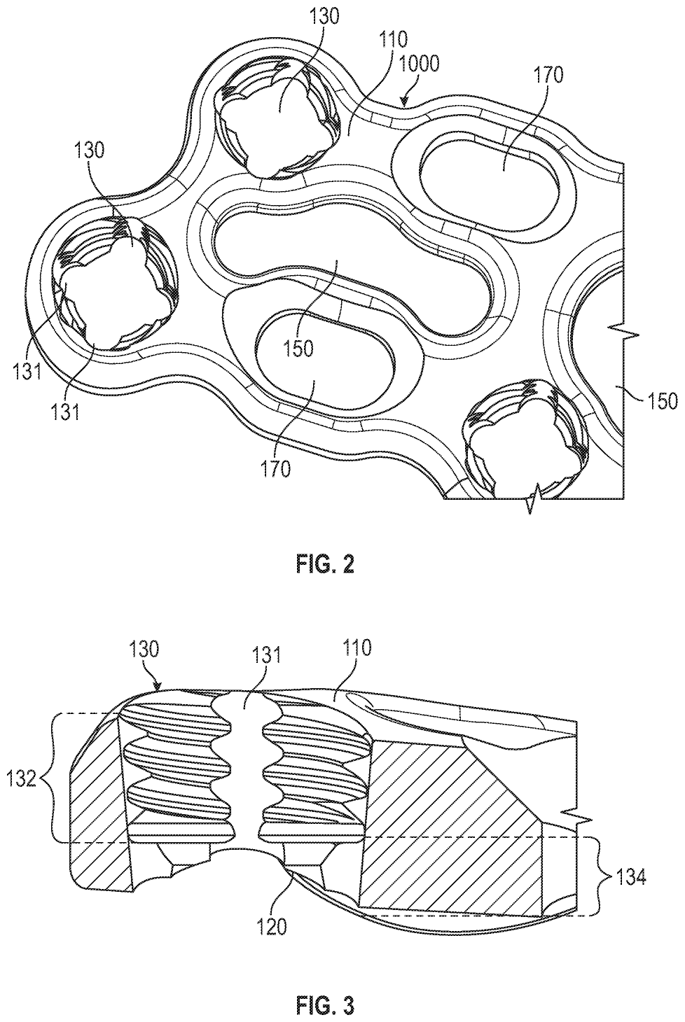

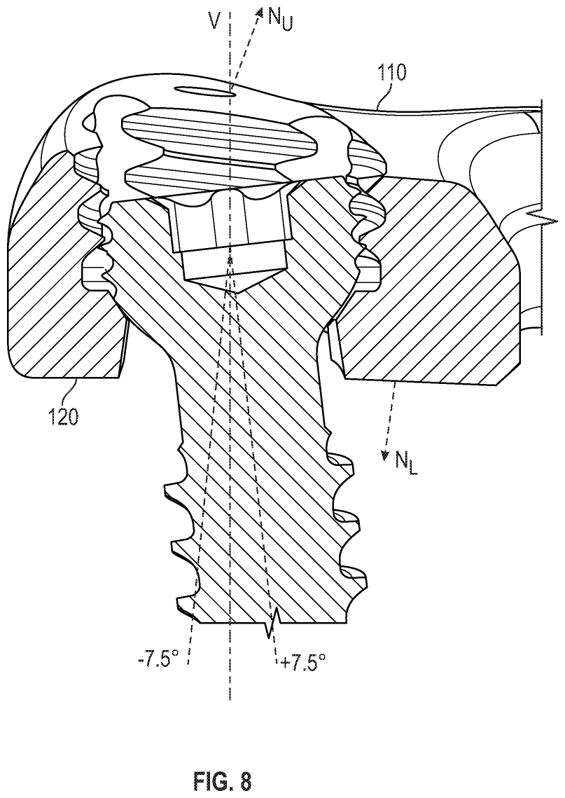

FIGS. 1 to 10 illustrate various views of a bone fixation system 1000 comprising a bone fixation plate 100 and one or more bone screws that have been generally denoted by the reference numeral 200. For the sake of clarity, only one bone screw 200 has been shown in the illustrations. However, it would be understood that each bone fixation plate 100 can be used with a plurality of bone screws 200 since each bone fixation plate 100 comprises a plurality of bone screw engaging passages 110 as will be evident from the foregoing sections.

The bone fixation plate 100 is contoured and dimensioned for application to a patients bone that needs to be reconstructed or treated. The bone fixation plate 100 comprises an in-use upper surface 110 and an in-use lower surface 120 (that directly engages with the bone during use) with respective openings 112 and 122 formed on the upper and lower surfaces 110 and 120 to form a passage 130 extending from the upper surface 110 to the lower surface 120 to engage a bone screw 200.

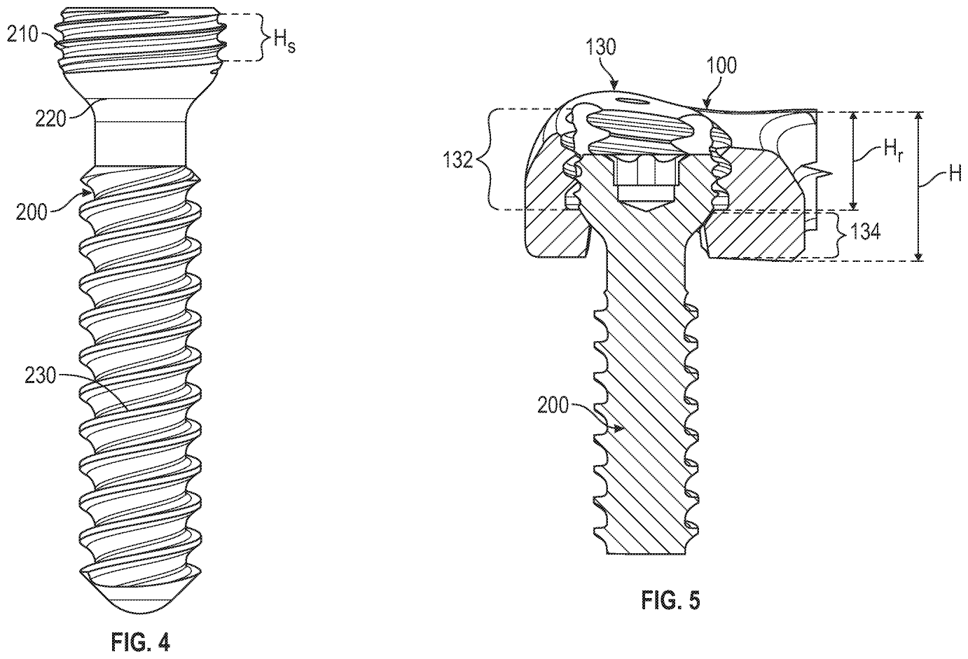

Referring to FIGS. 3 to 6, the passage 130 comprises novel structural characteristics which allows the bone screw 200 to function as a variable angle locking screw. Each passage 130 is defined by inner wall(s) extending from the upper surface 110 to the lower surface 120 with a heigh H extending between the upper surface 110 and the lower surface 120. The overall height of the inner wall for each passage can be split into a threaded upper inner wall portion 132 and a non-threaded lower inner wall portion 134. The threaded inner wall portion 132 extends from the upper surface 110 towards the lower surface 120 and tapers in a radially inward direction with threads having a thread pitch P_{W}. It is important to note that in at least some embodiments, the threaded inner wall portion 132 may not taper in the radially inward direction. The importance of the non-threaded lower inner wall portion 134 will be discussed in further detail in the foregoing sections.

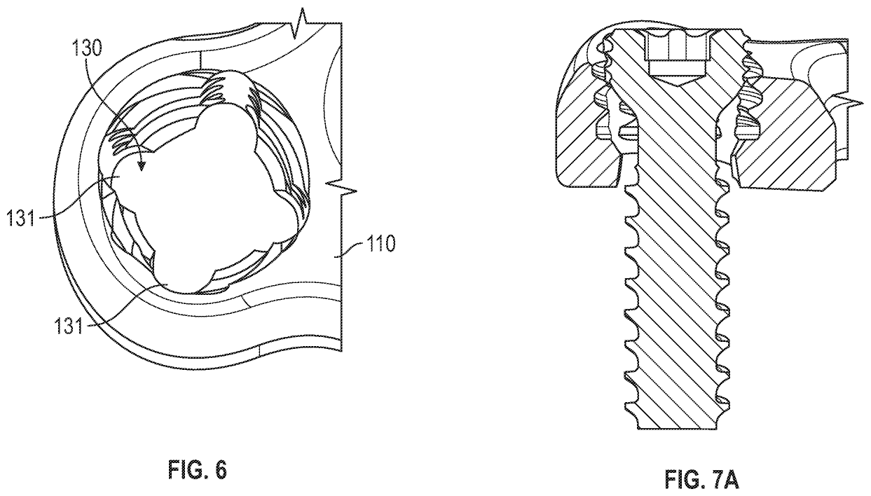

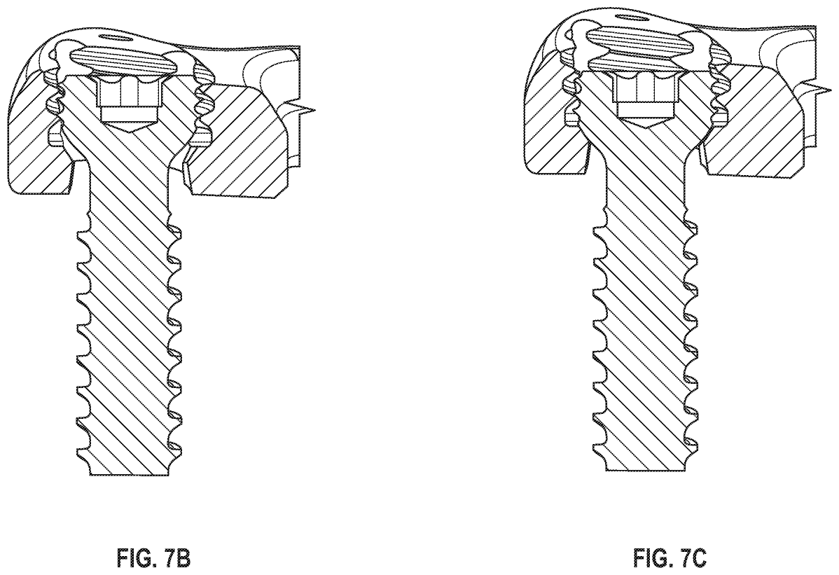

The bone screw 200 shown in FIGS. 4 and 5 comprises a threaded head 210 with threads having a pitch P_{H}. The bone screw 200 transitions from the enlarged head 210 via a transition zone 220 into a threaded shank 230. The height (H_{S}) of the threaded head 210 is less than the height H of the passage 130 and is preferably less than the height HT of the threaded wall section 132 (evident from FIG. 5). There is substantial thread pitch difference between the threaded head 210 of the bone screw 200 and the tapered threads provided on the threaded inner wall portion 132 of the bone fixation plate passage 130.

Specifically, the thread profile for the threaded screw head 210 is mismatched relative to thread profile of the threaded upper wall inner portion 132 for facilitating engagement between the threaded inner wall portion 132 and the threaded head 210 for effecting variable angle anchoring of the threaded screw head 210 during use. It is to be understood, that either the threads of the threaded inner wall portion 132 of the plate 100 or the threads of the threaded head 210 for the bone screw 200 may undergo sacrificial engagement which effects the variable angle locking. The gap between threads for the threaded inner wall region 132 is greater (preferably two times or more) than thickness of the threads for the threaded screw head 210 for facilitating sacrificial engagement of the threaded screw head 210 when interfacing with the inner wall portion 132 during use. The gap between threads for the threaded inner wall portion 132 is also greater than the gap between the threads for the threaded screw head 210 which facilitates engagement of the threads provided on the threaded screw head 210 when the threaded head 210 is advanced through the passage that allows variable angle locking. In addition, crest height for threads for the threaded upper inner wall portion 132 is greater than crest height for threads on the threaded head 210 of the bone screw 200.

Due to the enlarged gap between the threads of the screw head 210 in combination with the difference in thread gap and thread thickness between the threaded inner wall portion 132 and the threaded head 210 allows the screw 200 and specifically screw head 210 being inserted off-axis. In the example shown herewith, the thread pitch P_{W} for the threads on the inner wall portion 132 is greater than the thread pitch P_{H} of the threads for the threaded head 210 for the bone screw 200. As a result, the threads for the threaded head 210 in the bone screw 200 are likely to be sacrificially engaged when interfacing with the threads for the threaded inner wall portion 132 without resulting in the interlocking of the threads during advancement of the screw head 210 through the threaded inner wall portion 132. It should be understood and appreciated herein that those of skill in the art will be able to adjust the ratio of the gap-to-thread thickness or the pitch for the threads of either the screw head and/or the inner wall portion 132 to determine the maximum amount of off-axis screw angle.

The screw head 210 with the finer or smaller pitch functions as a sacrificial thread and it is hypothesized that these finer threads (with a smaller thickness) of the threaded head 210 in the bone screw 200 are stripped or cut by the thicker threads with the greater pitch provided on the threaded inner wall portion 132 as the bone screw head is advanced through the passage 130 (by applying torque on the screw head 210) thereby locking the bone screw 200 with the inner walls of the passage 130. In an alternative embodiment, the threaded inner wall region 132 may be provided with threads that have a smaller pitch relative to the pitch for the threads on the threaded head 210 of the bone screw 200. In such a scenario, it is expected that the threads for the inner wall section 132 are likely to be stripped away during engagement. It is somewhat preferable to have screws with a sacrificial head 210 and the illustrated embodiment, depicts a threaded head 210 for a bone screw 200 which comprises threads with a smaller pitch when compared with the threads for the threaded inner wall portion 132 of the bone fixation plate 100.

Referring to FIGS. 1, 2, 3 and 6, the passage 130 comprises a plurality of inner walls such that adjacently located inner walls, particularly adjacently located threaded inner wall portions 132 are separated by recessed channels 131 extending radially away from a central axis of the passage. In the presently described embodiment, there are four recessed channels 131 that separate adjacently located inner walls. These four recessed channels 131 comprise an outwardly curved surface and channels 131 are equi-angularly spaced relative to the central axis of the passage 130. It must be understood that the number of recessed channels 131 provided in each passage 130 may be varied and it not limiting in any manner. The equi-angular spacing of the recessed channels 131 is also not limiting. It is hypothesized that the recessed channels 131 function as relief cuts that promote the ability of the threads provided along the threaded upper inner wall portions 132 (located on either lateral side of each recessed channel 131) to cross thread and mismatch and adding locking ability to lock the threaded head 210 of the bone screw 200 with the inner wall portion 132 of the bone fixation plate 100.

As the bone screw head 210 advances through the passage 130 of the bone fixation plate 100, the relatively narrower non-threaded wall portion 134 of the passage engages with the non-threaded transition portion 220 of the bone screw 200 to stop and prevent further axial advancement of the bone screw 200. The non-threaded lower inner wall portion 134 also comprises a convergent bowl-shaped configuration to function as a hard stop for the bone screw 200 as the shank 230 of the bone screw 200 is advanced through the bone and as the threaded head 210 of the bone screw 200 is advanced through the height of the threaded wall portion 132. The non-threaded wall portion 134 comprises an upper portion that has a convergent shape with an overall varying width with an average width that is less than the width of the threaded screw head 210. It would also be understood that the opening provided by the non-threaded wall portion 134 allows the shank 230 to pass through and engage with the bone to carry out the intended function of the bone fixation system 1000.

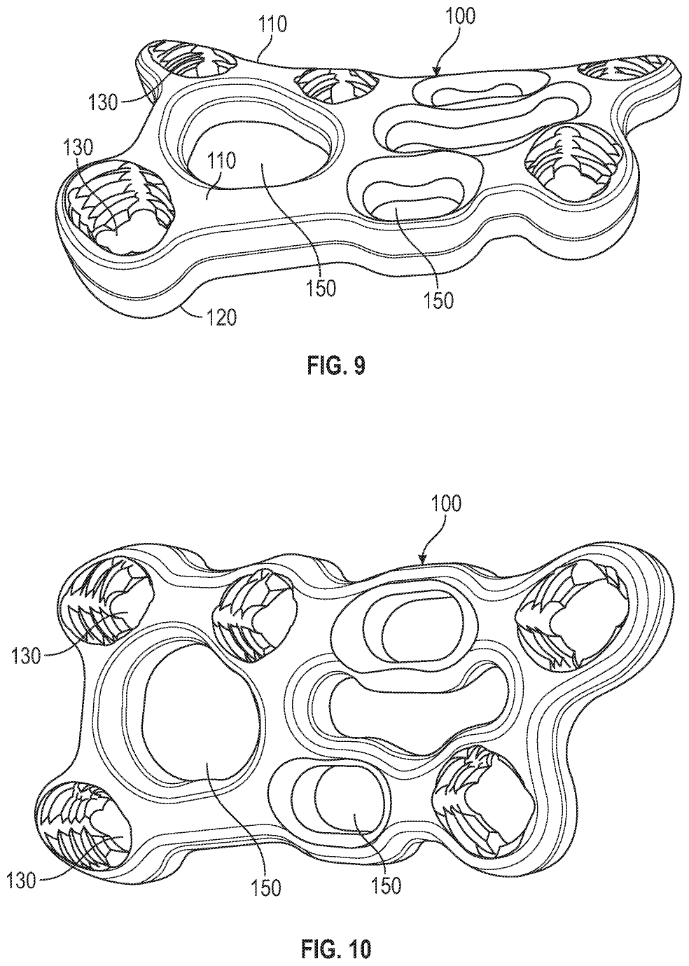

We now refer to FIGS. 7A to 7C to illustrate the working of the bone fixation plate system 1000 in further detail. Referring to FIG. 7A, in an initial step torque is applied to the bone screw 200, using a driver device, via a drive engagement interface on the threaded head 210. The threaded shank 230 engages the patient's bone. The thread profile provided on the shank 230 of the bone screw, a generic screw thread profile is provided. In the presently described embodiment a 1.2 mm thread pitch with a double start thread. In the initial step, the threaded screw head 210 engages with the threaded inner wall 132 of the passage once the shank 230 has been inserted substantially into the patient's bone. Referring to FIG. 7B, the threaded screw head 210 undergoes sacrificial engagement with the threaded inner wall 132 of the bone fixation plate 100. The difference in thread pitch between the threaded inner walls 132 (with the spaced apart relief recess channels 131) and the threads of the screw head 210 results in engagement and variable angle locking of the bone screw head 210 which in turn slows down the rate of advancement of the bone screw 200 into the bone of the patient. The mismatch between the threads of the bone screw head 210 and the threaded inner walls 132 results in compression between the bone fixation plate 100 and the patient's bone. As shown in FIG. 7C, the advancement of the screw head 210 has finally come to a stop because the transition portion 220 of the screw 200 is engaged with the convergent non-threaded lower wall portion 134 defining the lower portion of the passage 130.

We now refer to FIG. 8 that illustrates variable angle locking of the bone screw 210. The passage 130 comprises a vertical axis V and the novel configuration of the passage 130 (particularly the combination of the threaded upper wall portion 132 and the non-threaded lower wall portion 134 with the relief channels 131) as described in the preceding sections allows the bone screw 200 to be engaged by allowing the threaded screw head 210 to be engaged and locked into the passage 130 in a range of vertical orientations due to the mismatch between the thread pitch (P_{H}) and thread thickness of the threaded head 210 and the thread pitch (P_{W}) and thread thickness of the threaded wall portion 132. In the preferred embodiment, shown in FIG. 8, the tapered threads of the threaded wall portion 132 and the convergent configuration of the non-threaded wall portion 134 allows the bone screw to be inserted at an angle of ±7.5° from the vertical axis V. It is also important to note that upper surface 110 of bone fixation plate is 100 may not be flat and as a result the vertical axis (V) of the passage 130 may not be parallel to a normal axis (N_{U}) of the upper surface 110 or a normal axis NL of the lower surface 120 of the bone fixation plate 100. It is important to note that the vertical axis V substantially coincides with a central axis of the passage 130. The novel configuration of the bone fixation plate 100 is therefore particularly beneficial when the upper surface cannot be made flat for conforming with the anatomy of the patient's bone.

The bone fixation plate 100 may further include other non-threaded openings 150 extending between the upper and lower surfaces 110 and 120 and located in between two or more of the threaded passages 130. The non-threaded openings 150 have a somewhat irregular shape and function as relief holes and allow the bone fixation plate 100 to flex along preferred directions. The bone fixation plate 100 may also include other oval shaped non-threaded screw holes 170 for receiving additional screws.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1.-27. (canceled)

28. A bone fixation system for treatment of bones, the bone fixation system comprising:
a bone fixation plate configured and dimensioned for application to a patient's bone, the bone fixation plate comprising an in-use upper surface and an in-use lower surface with at least one opening formed on the upper and lower surfaces to form a passage extending from the upper surface to the lower surface;
the passage being defined by inner walls extending from the upper surface to the lower surface, the inner walls having a height extending between the upper surface and the lower surface, the inner wall further comprising:
a threaded upper inner wall portion extending from the upper surface towards the lower surface the threaded inner wall portion having threads with a thread pitch (P_{W});
a non-threaded lower inner wall portion extending from a lower part of the threaded inner wall portion towards the lower surface of the bone fixation plate; and
at least one bone screw with a threaded screw head having threads with a thread pitch (P_{H}) whereby thread profile for the threaded screw head is mismatched relative to thread profile of the threaded upper wall inner portion, wherein the mismatch comprises a gap between threads for the threaded inner wall portion, wherein the gap is greater than a thickness of the threads for the threaded screw head for facilitating engagement of the threaded screw head when interfaced with the threaded upper inner wall portion for effecting variable angle anchoring of the threaded screw head during use, and
wherein crest height for threads for the threaded upper inner wall portion is greater than crest height for threads on the threaded head of the bone screw.

29. A bone fixation system in accordance with claim 28 wherein the gap between the threads for the threaded inner wall portion is at least two times the thickness of the threads for the threaded screw head.

30. A bone fixation system in accordance with claim 28 wherein the gap between threads for the threaded inner wall portion is greater than the gap between the threads for the threaded screw head for facilitating engagement of the threaded screw head.

31. A bone fixation system in accordance with claim 28 wherein P_{W}>P_{H} to allow the threads on the screw head to engage with the threaded inner wall portion as the screw head is advanced through the passage of the bone plate.

32. A bone fixation system in accordance with claim 28 wherein the passage is defined by a plurality of said inner walls such that adjacently located inner walls are separated by a recessed channel extending radially away from a central axis of the passage.

33. A bone fixation system in accordance with claim 32 comprising N number of recessed channels and wherein the N recessed channels are angularly spaced at a distance of 360°/N relative to the central axis of the passage.

34. A bone fixation system in accordance with claim 32 wherein the recessed channels comprise a curved surface.

35. A bone fixation system in accordance with claim 28 wherein the lower inner wall portion defines a convergent configuration terminating in an opening with a width that is less than a width of the threaded head of the bone screw for preventing advancement of the screw head.

36. A bone fixation system in accordance with claim 28 wherein the lower inner wall portion comprises a convergent surface to prevent advancement of the screw head through the passage.

37. A bone fixation system in accordance with claim 28 wherein height of the passage is greater than height of the threaded head of the screw.

38. A bone fixation system in accordance with claim 28 wherein height of the threaded upper inner wall portion is greater than height of the threaded head of the bone screw.

39. A bone fixation system in accordance with claim 28 wherein threads for the threaded inner wall portion have an average thickness that is greater than an average thickness of the threads for the threaded head of the bone screw.

40. A bone fixation system in accordance with claim 28 wherein the passage comprises a vertical axis (V-V), wherein the threaded screw head is configured to engage with the threaded wall portion of the passage at an off-axis angle of up to 15 degrees and at least up to 5 degrees in any direction relative to the vertical axis.

41. A bone fixation system in accordance with claim 28 wherein the threaded upper inner wall portion tapers in a radially inward and downward direction.

42. A bone fixation system in accordance with claim 28 wherein the threads on the screw head are preferably sacrificially engaged with the threaded inner wall portion as the screw head is advanced through the passage of the bone plate.

43. A bone fixation system for treatment of bones, the bone fixation system comprising:
a bone fixation plate configured and dimensioned for application to a patient's bone, the bone fixation plate comprising an in-use upper surface and an in-use lower surface with at least one opening formed on the upper and lower surfaces to form a passage extending from the upper surface to the lower surface;
the passage being defined by inner walls extending from the upper surface to the lower surface, the inner walls having a height extending between the upper surface and the lower surface, the inner wall further comprising:
a threaded upper inner wall portion extending from the upper surface towards the lower surface the threaded inner wall portion having threads with a thread pitch (P_{W}); and
at least one bone screw with a threaded screw head having threads with a thread pitch (P_{H}) whereby thread profile for the threaded screw head is mismatched relative to thread profile of the threaded upper wall inner portion, wherein the mismatch comprises a gap between threads for the threaded inner wall portion, wherein the gap is greater than a thickness of the threads for the threaded screw head for facilitating engagement of the threaded screw head when interfaced with the threaded upper inner wall portion for effecting variable angle anchoring of the threaded screw head during use,
wherein crest height for threads for the threaded upper inner wall portion is greater than crest height for threads on the threaded head of the bone screw.

44. A bone fixation system in accordance with claim 43 wherein gap between the threads for the threaded inner wall portion is at least two times the thickness of the threads for the threaded screw head.

45. A bone fixation system in accordance with claim 43 wherein gap between threads for the threaded inner wall portion is greater than the gap between the threads for the threaded screw head for facilitating engagement of the threaded screw head.

46. A bone fixation system in accordance with claim 42 wherein P_{W}>P_{H} to allow the threads on the screw head to engage with the threaded inner wall portion as the screw head is advanced through the passage of the bone plate.

47. A bone fixation system in accordance with claim 42 wherein the passage is defined by a plurality of said inner walls such that adjacently located inner walls are separated by a recessed channel extending radially away from a central axis of the passage.
